(19) United States
(12) Patent Application Publication
(10) Pub. No.: US 2004/0009155 A1
(43) Pub. Date: Jan. 15, 2004

(54) Method for sustaining direct cell delivery

(76) Inventors: Maria Palasis, Wellesley, MA (US); James J. Barry, Marlborough, MA (US); Arthur Rosenthal, Boston, MA (US)

Correspondence Address: KENYON & KENYON, 1500 K STREET, N.W., SUITE 700, WASHINGTON, DC, 20005, US

(21) Appl. No.: 10/193,124

(22) Filed: Jul. 12, 2002

(51) Int. Cl. A61K 35/12

(52) U.S. Cl. 424/93.21; 424/93.7

(57) ABSTRACT

The present invention provides a method of sustaining direct delivery of cells into a target area. The method generally includes delivering cells to a target area. Specifically, the cells are introduced through an introduction site of a target area and delivered to the target area. The method further includes depositing at the introduction site, a plug member that contains a therapeutic agent that is released from the plug member to the target site. at the introduction site. The therapeutic agent generally acts to increase intracellular coupling between the grafted cells and the target area. The plug member also acts to seal the introduction site to inhibit cells from leaking from the introduction site into surrounding areas of the body.

### FIELD OF THE INVENTION

The present invention provides a method for sustaining the delivery of cells to a target area within a mammalian body.

### BACKGROUND OF THE INVENTION

Delivery of cells directly into tissue has been used to treat a variety of tissue disorders including damage to areas of the heart, brain, kidney, liver, gastrointestinal tract, and skin. Direct cell delivery (also referred to herein as “cellular transplantation” or “cellular graft”), as opposed to systemic delivery, has been considered to increase the density of cells in the target area and therefore increase cell survival in tissue, as it is believed that cells must form clusters to survive in tissue. Despite the increase in cell survival as a result of direct cell delivery versus systemic delivery, only a limited number of delivered cells survive post transplantation. In addition, scar tissue often forms in the target area separating the cellular graft from the target area. Furthermore, there is a potential for leakage of the delivered cells from the site of the target area through which the cells are introduced, further limiting the amount of cells that act in the target area. This problem is exacerbated in situations where the cells are injected into the tissue of an organ that undergoes expansion and contraction, such as the heart. In such cases, the organ wall thins during organ expansion, thus facilitating the leakage of previously injected cells from the target area and thereby decreasing the actual amount of cells delivered to the target area.

Therefore, a need exists for improved methods of direct cell delivery. The instant invention provides a method for direct and efficient delivery of cells that increases the number of injected cells that survive post transplantation and limits the formation of scar tissue that can separate the graft or transplant from the target area. The presently claimed method achieves these benefits, while also providing for inhibition of cell leakage from the introduction site of the target area.

### SUMMARY OF THE INVENTION

The present invention provides a method for sustaining the delivery of cells to a target area within a mammalian body. The method involves introducing cells through an introduction site of a target area and delivering the cells to the target area. The method further includes depositing at the introduction site, a plug member that contains a therapeutic agent that is released from the plug member to the target area.

The present invention provides another method for sustaining the delivery of cells to a target area within a mammalian body. The method involves introducing cells through an introduction site of a target area and delivering the cells to the target area. The method also includes applying an energy stimulus to the target area that promotes a wound-healing response in the target area. The method further includes depositing at the introduction site, a plug member that contains a therepeutic agent that is released from the plug member to the target area.

The present invention additionally provides a method for sustaining the delivery of cells to a target area within a body. The method involves introducing cells through an introduction site of a target area and delivering the cells to the target area. The method further includes applying an energy stimulus to the target area that promotes a wound-healing response in the target area.

### DETAILED DESCRIPTION OF THE INVENTION

The effectiveness of cell delivery or transplantation methods is often limited by the low survival rate of cells subsequent to transplantation as well as the formation of scar tissue, which can separate the cellular graft or transplant from the host target area. In general, the present invention provides a method of sustaining the delivery of cells to a target area within a mammalian body by depositing at the site through which the cells are introduced (also referred to herein as the “introduction site”), a plug member containing a therapeutic agent. The therapeutic agent is released to the target area and acts to generally increase intracellular coupling between the target area and the grafted cells. The target area may include any area of the body that is capable of accepting a cellular graft including for example, a tissue, an organ, or a blood vessel. Because the plug member is deposited at the site of cell introduction site, the plug member also inhibits cells from leaking from the introduction site into surrounding parts of the body and thereby prevents or precludes the need for repeated injection of cells to ensure effective and sustained treatment.

With respect to the particular details of the present invention, cells useful for the present invention include, but are not limited to, normal or genetically modified mesenchymal stem cells, hematopoietic stem cells, progenitor cells, cardiomyocytes, myoblasts, procardiomyocytes, skeletal fibroblasts, and pericytes. The type of cells selected for delivery will generally depend on the nature of the target area. For example, if the target area is a myopathic muscle, then myogenic cells may be used; if the target area is cartilage, then chondrocytes or fibroblasts may be selected; and if the target area is the myocardium then cardiomyoctes or skelatal myoblasts may be used. The cells used in the present invention can be obtained or isolated from any suitable source and can be isolated using techniques well known to the art. In general, the cells may be obtained from the recipient of the cells (autologous), from a donor of the same species as the recipient (homologous), or from a donor of a different species as the recipient (heterologous). Specifically, cells may be obtained from a tissue biopsy from one of the above-mentioned sources and the tissue biopsy may be digested with collagenase or trypsin, for example, to dissociate the cells. Alternatively, the cells may be obtained from bone marrow or peripheral blood. The cells may also be obtained from established cell lines or from embryonic cell sources.

The cells useful in the present invention may be delivered to the target area via any suitable manner known in the art of direct delivery including engraftment, transplantation, or direct injection via a needle or catheter. Examples of specific devices incorporating injection needles include needle injection catheters, hypodermic needles, biopsy needes, ablation catheters, cannulas and any other type of medically useful needle. Examples of non-needle injection direct delivery devices include, but are not limited to, transmural myocardial revascularization (TMR) devices and percutaneous myocardial revascularization (PMR) devices.

The plug member according to the present invention comprises a biocompatible member containing a therapeutic agent that generally functions to increase intracellular coupling between the transplanted cells and the target area. The therapeutic agent released by the plug member to the target area may comprise any protein, pharmaceutically active compound, nucleic acid (including anti-sense DNA and RNA; DNA coding for an anti-sense RNA; or DNA coding for tRNA), peptide, polypeptide, lipid, carbohydrate, small molecule, hormone, coenzyme and metabolite, amino acid, virus, metal including an organic and organometallic compound and salts thereof, polymer, or any combination thereof that operates to enhance the efficiency of cell delivery and/or increase the effectiveness of cell engraftment, survival, adhesion, division, or any combination thereof. For example, as the survival rate of cells is low subsequent to cellular transplant, the therapeutic agent may be an agent that functions to prevent apoptosis or increase the survival of injected cells. Non-limiting examples of such an agent include heat shock proteins, and anti-apoptotic agents such as AKT and NF-kB. In another embodiment, as scar tissue may form at the target area, which separates the cellular graft or transplant from the target area, the therapeutic agent comprises an agent that increases cell engraftment efficiency and cell adhesion. Non-limiting examples of such an agent include integrins, N-cadherin, and connexin-43. The therapeutic agent may also include any substance that increases the survival of cells and promotes cell engraftment, for example, by increasing cell division or promoting angiogenesis. Non-limiting examples of such agents include growth factors such as vascular endothelial growth factor, hepatocyte growth factor, epidermal growth factor, transforming growth factor α and β₂, platelet-derived endothelial growth factor, platelet-derived growth factor, tumor necrosis factor alpha, insulin growth factor and acidic and basis fibroblast growth factor; and angiopoietins such as H1F1, Del1, PR39, or NF-kB. In addition, as cell transplants, particularly homologous cell transplants, may evoke a strong immune response, in another embodiment, the therapeutic agent may also include substances that suppress inflammation or an immune response such as cyclosoporin, and cytokines such as interleukins and lymphokines.

One skilled in the art would appreciate that the type of therapeutic agent to be released also depends on the type of cells delivered to the target area and the desired outcomes of the cell transplantation. For example, if the target area is nerve tissue and nerve cells and support cell are injected into the nerve tissue, then the therapeutic agent may comprise nerve growth factor. If the target area is bone or connective tissue and osteocytes and periosteal cells are injected to the bone or connective tissue, then the therapeutic agent may comprise insulin-growth factor or bone growth factor such as BMP-7, BMP-2, BMP-2, BMP-4, BMP-5, BMP-6, BMP-7, BMP-8, BMP-9, BMP-10, BMP-11, BMP-12, BMP-13, BMP-14, BMP-15, and BMP-16.

With respect to the properties of the plug member, the plug member may be fabricated of a non-polymeric material or, preferably a polymeric material. In general, the plug member may be fabricated of any material capable of delivering a therapeutic agent to a target area and capable of sealing the introduction site so as to inhibit or prevent cell leakage. In particular, the plug member may be composed of biodegradable polymers such as, for example, ethylene vinyl acetate, polyanhydrides, polyglycolic acid, polyorthoesters, polyactic acid, polycarboxylic acids, cellulose polymers, including cellulose acetate and cellulose nitrate, gelatin, polyvinylpyrrolidone, cross-linked polyvinylpyrrolidone, hydrogels, polyanhydrides including maleic anhydride polymers, polyamides, polyvinyl alcohols, copolymers of vinyl monomers such as EVA, polyvinyl ethers, polyvinyl aromatics, polyethylene oxides, glycosaminoglycans, polysaccharides, ethylene vinylacetate, polyesters including polyethylene terephthalate, polyacrylamides, polyethers, polyether sulfone, polycarbonate, polyalkylenes including polypropylene, polyethylene and high molecular weight polyethylene, halogenated polyalkylenes including polytetrafluoroethylene, polyurethanes, polyorthoesters, proteins, polypeptides, silicones, silocones siloxane polymers, polylactic acid, polyglycolic acid, polycaprolactone, polyhydroxybutyrate valerate and blends and copolymers thereof as well as other biodegradable, bioabsorbable and biostable polymers and copolymers.

By “biodegradable” is meant that the material of the plug member will degrade over time by the action of enzymes, by simple or enzymatically catalyzed hydrolytic action and/or by other similar mechanisms in the human body. By “bioabsorbable,” is meant that the material of the plug member will be broken down and absorbed within the human body, for example, by a cell, a tissue, and the like.

The plug member may also be made of non-biodegradable materials such silicons, glasses, silicone elastomers. Further examples include sintered hydroxyapatite, bioglass and aluminates, for example.

In certain embodiments, the plug member is composed of a non-polymeric material that is combined with at least one organic solvent. The organic solvent is biocompatible and will at least partially dissolve the non-polymeric material. Preferably, the organic solvent has a solubility in water ranging from miscible to dispersible. The solvent is capable of diffusing, dispersing, or leaching from the non-polymeric composition in situ into aqueous tissue fluid of the target area such as blood serum, lymph, cerebral spinal fluid (CSF), saliva, and the like.

Solvents that are useful include, for example, substituted heterocyclic compounds such as N-methyl-2-pyrrolidone (NMP) and 2-pyrrolidone (2-pyrol); esters of carbonic acid and alkyl alcohols such as propylene carbonate, ethylene carbonate and dimethyl carbonate; fatty acids such as acetic acid, lactic acid and heptanoic acid; alkyl esters of mono-, di-, and tricarboxylic acids such as 2-ethyoxyethyl acetate, ethyl acetate, methyl acetate, ethyl lactate, ethyl butyrate, diethyl malonate, diethyl glutonate, tributyl citrate, diethyl succinate, tributyrin, isopropyl myristate, dimethyl adipate, dimethyl succinate, dimethyl oxalate, dimethyl citrate, triethyl citrate, acetyl tributyl citrate, glyceryl triacetate; alkyl ketones such as acetone and methyl ethyl ketone; ether alcohols such as 2-ethoxyethanol, ethylene glycol dimethyl ether, glycofurol and glycerol formal; alcohols such as ethanol and propanol; polyhydroxy alcohols such as propylene glycol, polyethylene glycol (PEG), glycerin (glycerol), 1,3-butyleneglycol, and isopropylidene glycol; dialkylamides such as dimethylformamide and dimethylacetamide; dimethylsulfoxide (DMSO) and dimethylsulfone; tetrahydrofuran; lactones such as e-caprolactone and butyrolactone; cyclic alkyl amides such as caprolactam; aromatic amides such as N,N-dimethyl-m-toluamide, and 1-dodecylazacycloheptan-2-one; and the like; and mixtures and combinations thereof. Preferred solvents include N-methyl-2-pyrrolidone, 2-pyrrolidone, dimethylsulfoxide, ethyl lactate, propylene carbonate, glycofurol, glycerol, and isopropylidene glycol. Preferably the organic solvent is non-toxic.

A composition of the non-polymeric material is preferably flowable with a consistency that ranges from watery to slightly viscous to a putty or paste. The non-polymeric composition eventually coagulates to a microporous, solid matrix upon the dissipation of the organic solvent into adjacent tissue fluids. The non-polymeric composition can be manipulated and shaped within the target area as it solidifies. Advantageously, the moldability of the composition as it hardens allows it to conform to irregularities, crevices, cracks, holes, and the like, in the introduction site of the target area. The resulting substantially solid matrix is preferably biodegradable, bioabsorbable, and/or bioerodible, and will be gradually absorbed into the surrounding tissue fluids, and become disintegrated through enzymatic, chemical and/or cellular hydrolytic action. By “bioerodible” is meant that the plug member will erode or degrade over time due, at least in part, to contact with substances found in the surrounding tissue fluids, cellular action, and the like.

Optionally, the composition of non-polymeric material of this embodiment can be combined with a minor amount of a biodegradable, bioabsorbable thermoplastic polymer such as a polylactide, polycaprolactone, polyglycolide, or copolymer thereof, to provide a more coherent solid plug member or a composition with greater viscosity so as to hold the plug member in place while it solidifies. The non-polymeric materials are also capable of coagulating or solidifying to form a solid plug member upon the dissipation, dispersement or leaching of the solvent component from the composition and contact of the non-polymeric material with an aqueous medium. The solid plug member has a firm consistency ranging from gelatinous to impressionable and moldable, to a hard, dense solid.

Non-polymeric materials according to this embodiment that are suitable for use in the present invention generally include any having the foregoing characteristics. Examples of useful non-polymeric materials include sterols such as cholesterol, stigmasterol, β-sitosterol, and estradiol; cholesteryl esters such as cholesteryl stearate; C₁₂-C₂₄ fatty acids such as lauric acid, myristic acid, palmitic acid, stearic acid, arachidic acid, behenic acid, and lignoceric acid; C₁₈-C₃₆ mono-, di- and triacylglycerides such as glyceryl monooleate, glyceryl monolinoleate, glyceryl monolaurate, glyceryl monodocosanoate, glyceryl monomyristate, glyceryl monodicenoate, glyceryl dipalmitate, glyceryl didocosanoate, glyceryl dimyristate, glyceryl didecenoate, glyceryl tridocosanoate, glyceryl trimyristate, glyceryl tridecenoate, glycerol tristearate and mixtures thereof; sucrose fatty acid esters such as sucrose distearate and sucrose palmitate; sorbitan fatty acid esters such as sorbitan monostearate, sorbitan monopalmitate and sorbitan tristearate; C₁₆-C₁₈ fatty alcohols such as cetyl alcohol, myristyl alcohol, stearyl alcohol, and cetostearyl alcohol; esters of fatty alcohols and fatty acids such as cetyl palmitate and cetearyl palmitate; anhydrides of fatty acids such as stearic anhydride; phospholipids including phosphatidylcholine (lecithin), phosphatidylserine, phosphatidylethanolamine, phosphatidylinositol, and lysoderivatives thereof; sphingosine and derivatives thereof; spingomyelins such as stearyl, palmitoyl, and tricosanyl spingomyelins; ceramides such as stearyl and palmitoyl ceramides; glycosphingolipids; lanolin and lanolin alcohols; and combinations and mixtures thereof. Preferred non-polymeric materials include cholesterol, glyceryl monostearate, glycerol tristearate, stearic acid, stearic anhydride, glyceryl monooleate, glyceryl monolinoleate, and acetylated monoglycerides. Further examples of the composition of the plug member are discussed below in conjunction with the fabrication of the plug member.

As is known to one of skill in the art, the composition of the plug member may depend on the manner of fabrication of the plug member. With respect to the fabrication of the plug member, the plug member may be pre-formed prior to use or formed by in situ phase change or in situ polymerization. In one embodiment, the plug member is heated (or cooled, depending on the temperature at which the material of the plug member being used is in the liquid phase) prior to application to the introduction site and subsequent cooled (or heating) to solidify the plug member and seal the introduction site. For example, a temperature sensitive polymer, which is liquid at above or below physiological temperature (i.e. about 37° C.) and solidifies at physiological temperature may be used in this embodiment. Examples of suitable materials for use in this embodiment include N-isopropylacrylamide and certain celluloses.

In another embodiment, the plug member is applied to the introduction site while the plug member is in a first fluent state. Then the plug member is maintained in a position so as to plug the introduction site under conditions that convert the plug member in situ into a second less-fluent or essentially non-fluent state. The conversion may be achieved either by changing the environment surrounding the member, by the addition or removal or chemicals or energy, or by passive means such as maintaining the plug member at the normal internal body temperature of a patient. The transition of the state of the plug member from a fluent state to a less fluent state or essentially non-fluent state may be the result of a phase change, viscosity change, or polymerization.

In embodiments where the plug member is formed by in situ polymerization or phase change, the plug member is fabricated of a polymer that can be polymerized or have its viscosity altered in vivo preferably by the application of light, ultrasound, radiation, or chelation, alone or in the presence of added catalyst or divalent ions, or by a change to physiological pH, or change in temperature to body temperature.

Non-limiting examples of such polymers that alter viscosity as a function of temperature include poly(oxyalkene) polymers and copolymers such as poly(ethylene oxide) -poly(propylene oxide) copolymers, and copolymers and blends of these polymers with polymers such as poly(alpha-hydroxy) acids, including but not limited to lactic, glycolic, and hydroxybutyric acids, polycarprolactones, and polyvalerolactones.

Examples of polymers that polymerize in the presence of divalent ions such as calcium, barium, magnesium, copper, and iron include naturally occurring polymers collagen, fibrin, elastin, agarose, agar, polysaccharides such as hyaluronic acid, hyalobiuronic acid, heparin, cellulose, alginate, curdlan, chitin, and chitosan, and derivatives thereof, cellulose acetate, carboxymethyl cellulose, hydroxymethyl cellulose, cellulose sulfate sodium salt, and ethylcellulose.

Examples of polymers that can be cross-linked photochemically with ultrasound or with radiation generally include those polymers that contain a double-bond or a triple-bond. Examples of such polymers include monomers that are polymerized into poly(acrilic acids), poly(acrylates), polyacrylamides, polyvinyl alcohols, polyethylene glycols, and ethylene vinyl acetates.

As is generally known to one skilled in the art, the release rate of the therapeutic agent may be controlled by the particular composition of the plug member. For example, where the plug member is made of polymeric material, the amount, concentration, and type of polymer can control release of agents and where the plug member is composed of a biodegradable material, the release of agents contained in a biodegradable plug can be controlled by the rate of degradation of the plug member. Preferably, the plug member is fabricated to provide for sustained release of the therapeutic agents. In such an embodiment, the plug member may be composed of styrene isobutylene styrene (SIBS), including styrene and isobutyl styrene, which may not readily degrade within the body and therefore may be used for sustained delivery of the therapeutic agent to the target site. As is known to one of skill in the art, the release rate of the therapeutic agent may also be adjusted by modifying various properties of the plug member as well as by adjusting the properties of the therapeutic agent relative to the plug member. These properties include the porosity of the plug member, which may be affected by adjusting the pressure placed on material being forced through nozzle during the manufacturing process to create the plug member. The properties also include the size, shape, dosage form, and quantity of the therapeutic agent.

The size of the plug member should preferably be such that the plug member can substantially seal the site of cell introduction to prevent or reduce cell leakage. The size of the plug member will therefore vary depending on the mode of administration. For example, if cells are delivered to the target site via a needle, then the diameter of the plug member should at least substantially correspond to the diameter of the needle in order to properly seal the opening created by the needle at the introduction site. The plug member may also be deposited at the introduction site by any suitable means of administration such as, for example, direct injection via a needle or catheter and may be deposited after or during cell delivery. The plug members may also be delivered through a lumen of a multi-lumen catheter, in which case the cells are delivered via a separate lumen.

The plug member may optionally include a bioadhesive that is released to the target area. Bioadhesive material may be any biocompatible additive that results in an increase of the affinity of the transplanted cells to the target area. Bioadhesive materials for use in conjunction with the invention include suitable bioadhesive materials known to those of ordinary skill in the art. By way of example, suitable bioadhesive materials include fibrinogen, with or without thrombin, fibrin, fibropectin, elastin, laminin, cyanoacrylates, polyacrylic acid, polystyrene, bioabsorbable and biostable polymers derivitized with sticky molecules such as arginine, glycine, and aspartic acid, and copolymers.

With respect to uses of the present invention, in one embodiment, cells are introduced through an introduction site of a target area of the body. The cells are delivered to the target area and a plug member is subsequently or concurrently deposited at the site of introduction. The cells and plug member may be introduced and delivered through the same instrument or different instruments. For example, the cells may be introduced and delivered via an injection needle and then withdrawn from the introduction site. The plug member may then be deposited via a different injection needle to the introduction site. Alternatively, a dual-lumen catheter may be utilized with the cells placed in one lumen and the plug member inserted in the other lumen. The cells and plug member may then be concurrently delivered to the target area. Nothwithstanding the method of delivery, the plug member releases therapeutic agents in the target area that act to generally increase intracellular coupling between the target area and the grafted cells by, for example, increasing cell adhesion or preventing apoptosis.

In another embodiment, the plug member is used to regulate the expression of biomolecules by delivered cells. These biomolecules include any substance that enhances the efficiency of cell delivery and/or increases the effectiveness of cell engraftment, survival, adhesion, division, or any combination thereof. The biomolecules include for example, proteins including structural, chimeric and fusion proteins; peptides; saccharides; oligosaccharides; polysaccharides; oligopeptides; polypeptides; oligonucleotides and polynucleotides (e.g., DNA, cDNA, dsDNA, ssDNA); aminio acids; nucleotides; lipids; carbohydrates; hormones; coenzymes; and specifically growth factors; cytokines; and pharmaceutically active compounds. In this embodiment, the cells may be genetically transformed prior to delivery to the target area in order to express or over-express a particular biomolecule.

Methods for transforming cells are known in the art and generally involve transfecting the cells with genes encoding the biomolecule of interest. The cells may be transfected using any appropriate means including viral vectors, chemical transfectants, or physio-mechanical methods such as electroporation and direct diffusion of DNA. If a vector is utilized, the vector includes the desired gene operably linked to a inducible promoter, which yields expression of the gene in the cells into which it is delivered after being activated by an inducing agent. Any suitable inducible promoter and corresponding suitable inducing agent is contemplated by the present invention. Exemplary inducible promoters include sheep metallothionine (MT) promoter, mouse mammary tumor virus (MMTV), or the tet promoter. Inducible promoters can be general inducible promoters, yielding expression in a variety of mammalian cells, or cell specific, or even nuclear versus cytoplasmic specific.

After the cells are transfected with the genes encoding the biomolecules of interest, the cells are delivered to the target area. A plug member containing a suitable inducing agent is then deposited at the introduction site and the inducible agent is released to the target area to activate the inducible promoter associated with the transfected delivered cells. Exemplary inducing agents include, glucocorticoids such as dexamethasone for the MMTV promoter, or a metal such as zinc for the MT promoter, or an antibiotic such as tetracycline for the tet promoter. After the inducible promoter is activated by the inducing agent, the expresson of the desired gene is “turned on” and the desired biomolecule is produced by the transfected cell. This embodiment allows for highly controlled expression of the cells, allowing for varying levels of expression of the delivered biomolecule. Use of an inducible promoter also allows for control on the timing of production of the biomolecule. Utilizing the plug member in this way also allows for expression of the gene only for the period of time that the inducing agent is released from the plug member. In this embodiment, upon depletion of such inducing agent from the plug member, the desired gene of the tranfected delivered cells is no longer expressed. For example, an anti-apoptic gene could be expressed only for the length of time required to insure cell engraftment.

In another embodiment of the present invention, the cells may also be transformed to express at least one enzyme which reacts with a prodrug contained within the plug member. A prodrug is generally defined as an inactive derivative of a drug molecule that requires a transformation, such as, for example, chemical or enzymatic, in order to release the active parent drug. In this embodiment, the transformed cells are delivered to the target area and concurrently or subsequently, the plug member containing the prodrug is deposited at the introduction site and the prodrug is released to the target area. In this embodiment, the enzyme expressed by the cells will transform and ‘activate’ the prodrug contained within the plug member. The active parent drug then interacts with the transplanted cells at the target area. The active prodrug can possess various functions, which include, but are not limited to, prevention of apoptosis, promotion of cell adhesion and angiogenesis, increase of cell engraftment and cell division, and suppression of abnormal inflammation or immune responses, or any combination thereof

In yet another embodiment, the application of an energy stimulus may be used in conjunction with the cell delivery methods of the present invention. The application of an energy stimulus also stimulates the wound-healing response and therefore increases tissue permeability and thus may increase cell migration and/or engraftment. The energy stimulus may also seal the introduction site by sealing the open ends of the tissue that characterize the introduction site. Non-limiting examples of energy stimuli that may be used in the present invention include ultrasound, lasers, radio-frequency, electrical current, or heating. For example, in one embodiment, the introduction site is sealed by performing radio frequency cautery at the introduction site to seal the introduction site. Cauterization involves using such intense heat to seal the open ends of the tissue. Radio frequency cautery may be performed by any suitable method known to those skilled in the art.

In another embodiment, the introduction site is sealed by performing laser heating at the introduction site to seal the introduction site. In this embodiment, laser emitted optical energy may be used to heat biological tissue to a degree suitable for denaturing the tissue proteins such that the collagenous elements of the tissue form a “biological glue” to seal the target area. Suitable methods of laser heating a tissue are known to those of skill in the art. Notwithstanding what type of energy stimulus is applied to the introduction site, the energy stimulus may be used in conjunction with cell delivery alone or may be used in conjunction with cell delivery and plug member deposit to complement the effect of the therapeutic agent released by the plug member. For example, cells may be delivered to the target area, an energy stimuli may be applied to the target area, and, optionally, a plug member containing a therapeutic agent may be deposited at the introduction site, wherein the therapeutic agent is released to the target area. The present invention is not limited to any particular order of these steps and the energy stimulus may be applied concurrent or subsequent to cell delivery and the plug member may be deposited concurrent or subsequent to application of the energy stimuli.

The instantly claimed plug member and methods of cell delivery can be utilized to treat and/or prevent disorders or diseases associated with any mammalian tissue or organ, whether injected in vivo or ex vivo including, for example, heart, lung, brain, liver, skeletal muscle, smooth muscle, kidney, bladder, intestines, stomach, pancreas, ovary, prostrate, eye, tuorms, cartilage, bone, and skin. Such disorders include, but are not limited to, angiogenic diseases, heart failure, myocardial ischemia, angina pectoris, myocardial infarction, stroke, amyotrophic lateral sclerosis (ALS), myastenia gravis, Eaton-Lambert Syndrome, muscular dystrophies, Parkinson's disease, Alzheimer's disease, type 1 diabetes or insulin-requiring type 2 diabetes.

We claim:

1. A method for sustaining the delivery of cells to a target area within a mammalian body comprising:
introducing cells through an introduction site of a target area;
delivering the cells to the target area; and
depositing a plug member at the introduction site, the plug member comprising a therapeutic agent that is released from the plug member to the target area.

2. The method of claim 1, wherein the therapeutic agent increases the survival rate of cells subsequent to the delivery of cells to the target area.

3. The method of claim 1, wherein the therapeutic agent inhibits the formation of scar tissue at the target area.

4. The method of claim 1, wherein the therapeutic agent inhibits apoptosis of the cells.

5. The method of claim 1, wherein the therapeutic agent promotes cell adhesion.

6. The method of claim 1, wherein the therapeutic agent increases cell division.

7. The method of claim 1, wherein the therapeutic agent promotes angiogenesis.

8. The method of claim 1, wherein the therapeutic agent suppresses inflammation.

9. The method of claim 1, wherein the therapeutic agent is a prodrug, and the cells are genetically transformed to express an enzyme that reacts with the prodrug.

10. The method of claim 1, wherein the cells are transformed with an inducible promotor operably linked to a gene encoding a biomolecule of interest, and the therapeutic agent is an inducing agent that activates the promotor.

11. The method of claim 10, wherein the biomolecule of interest increases intercellular coupling between the cells and the target area.

12. The method of claim 1, wherein the cells are selected from the group consisting of mesenchymal stem cells, hematopoietic stem cells, progenitor cells, cardiomyocytes, myoblasts, procardiomyocytes, skeletal fibroblasts, and pericytes.

13. A method of sustaining the delivering cells to a target area in a mammalian body comprising:
introducing cells through an introduction site of a target area;
delivering cells to the target area;
depositing a plug member at the introduction site of the cells, the plug member comprising a therapeutic agent that is released from the plug member to the target area; and
applying an energy stimulus to the target area, the energy stimulus promoting a wound-healing response in the target area.

14. The method of claim 13, wherein the therapeutic agent increases intercellular coupling between the cells and the target area.

15. A method of sustaining the delivering cells to a target area in a mammalian body comprising:
introducing cells through an introduction site of a target area;
delivering cells to the target area; and
applying an energy stimulus to the target area, the energy stimulus promoting a wound-healing response in the target area.
